(19) United States
(12) Patent Application Publication
(10) Pub. No.: US 2014/0135868 A1
(43) Pub. Date: May 15, 2014

(54) NON-INVASIVE INTRAORAL ELECTRICAL STIMULATOR SYSTEM AND METHOD FOR TREATMENT OF OBSTRUCTIVE SLEEP APNEA (OSA)

(71) Applicant: JACOB BASHYAM BASHYAM, LAGUNA NIGUEL, CA (US)

(72) Inventor: JACOB BASHYAM BASHYAM, LAGUNA NIGUEL, CA (US)

(21) Appl. No.: 14/074,694

(22) Filed: Nov. 7, 2013

Related U.S. Application Data
(60) Provisional application No. 61/724,881, filed on Nov. 9, 2012.

(51) Int. Cl. A61N 1/36; A61N 1/372

(52) U.S. Cl. CPC A61N 1/36003; A61N 1/37235
USPC 607/42

(57) ABSTRACT

A non-invasive, removable intraoral electrical Stimulator or Pacemaker system and method is described, for electrically-stimulating and re-establishing the tone in the upper pharyngeal dilator muscle, the genioglossus and base-of-tongue muscles, for the treatment of Obstructive Sleep Apnea (OSA) in human adults and young adults. The Stimulator system consists of an intraoral Stimulator device assembly with a rechargeable battery, an external (inductive) Recharger appliance and an external hand-held (inductive) Programmer appliance. The Stimulator device assembly is inserted into the mouth by the OSA patient before sleep time and is removed when awake or during normal activity and placed in the charging cradle of the Recharger appliance for recharging the device battery. The physician uses the hand-held Programmer appliance to determine and set the patient-specific stimulation therapy parameters in the device, at the patient's initial evaluation. The stimulation therapy is delivered either in an open loop configuration without regard to patient's respiration activity, or in a closed loop configuration synchronized to the patient's respiration detected by one or more sensors in the system.

### CROSS-REFERENCES TO RELATED APPLICATIONS

This application claims the benefit of under 35 U.S.C. §109(e) of U.S. Provisional Patent Application No. 61/724,881 filed on Nov. 9, 2012, the disclosure of which is incorporated herein by reference.

### STATEMENT AS TO RIGHTS TO INVENTIONS MADE UNDER FEDERALLY SPONSORED RESEARCH AND DEVELOPMENT.

Not Applicable

### REFERENCE TO A “SEQUENCE LISTING”, A TABLE, OR A COMPUTER PROGRAM LISTING APPNEDIX SUBMITTED ON A COMPACT DISK

Not Applicable

### OTHER REFERENCE PUBLICATIONS

1. E. F. Bailey and R. F. Fregosi, Department of Physiology, College of Medicine, University of Arizona, Tucson, Ariz., Coordination of intrinsic and extrinsic tongue muscles.
2. Bill Pruitt, A Resurrected Approach for Treating OSA, New found interest in hypoglossal nerve stimulation devices, Sleep Review January/February 2012, The Journal for Sleep Specialists.
3. H. Bishara, M. Odeh, R.P. Schnall, N. Gavriely, A. Oliven, Electrically-activated dilator muscles reduce pharyngeal resistance in anaesthetized dogs with upper airway obstruction, Eur Respir J, 1995, 8, 1537-1542.
4. Blumen M B, de La Sota A P, Quera-Salva M A, Frachet B, Chabolle F, Lofaso F., Tongue mechanical characteristics and genioglossus muscle EMG in obstructive sleep apnea patients. Respir Physiol Neurobiol. 2004 May 20; 140(2):155-64.
5. S. Cheng, J. E. Butler, S. C. Gandevia and L. E. Bilston, Movement of the tongue during normal breathing in awake healthy humans, J Physiol 586.17 (2008) pp 4283-4294.
6. David W. Eisele, MD et al., Direct Hypoglossal Nerve Stimulation in Obstructive Sleep Apnea, Arch Otolaryngol Head Neck Surg. 1997; 123(1):57-61.
7. R B Fogel, Sleep 2: Pathophysiology of obstructive sleep apnea/hypopnea syndrome, Thorax 2004; 59:159-163 doi:10.1136/thorax.2003.015859
8. Joerg Steier , M D, PhD; John Seymour , PhD, et al., Continuous Transcutaneous Submental Electrical Stimulation in Obstructive Sleep Apnea, http://journal.publications.chestnet.org/9.
9. Mann, E. A., Burnett, T., Cornell, S. and Ludlow, C. L. (2002), The Effect of Neuromuscular Stimulation of the Genioglossus on the Hypopharyngeal Airway. The Laryngoscope, 112: 351-356.
10. Michael J. Brennick, Warren B. Gefter, Susan S. Margulies, Mechanical effects of genioglossus muscle stimulation on the pharyngeal airway by MRI in cats, Respiratory Physiology & Neurobiology 156 (2007) 154-164.
11. Oliven et al, European Respiratory Journal published online Jun. 13, 2007, Effect of genioglossus contraction on pharyngeal lumen and airflow in sleep apnea patients.
12. Schwartz A R, Gold A R, Schubert N, Stryzak A, Wise R A, Permutt S, Smith P L., Effect of weight loss on upper airway collapsibility in obstructive sleep apnea, Am Rev Respir Dis. 1991 September; 144(3 Pt 1):494-8.
13. Tran, W. H. et al., Dept. of Biomedical Eng., Univ. of Southern California, Los Angeles, Calif., USA, Development of asynchronous, intralingual electrical stimulation to treat obstructive sleep apnea
14. Walsh J, Maddison K, Tesfayesus W, Hillman D, Eastwood P, Genioglossus muscle stimulation for the treatment of OSA. Sleep and Biological Rhythms 2009; 7(suppl 1):A2.

### BACKGROUND OF THE INVENTION

Sleep Apnea is a sleep disorder characterized by frequent abnormal pauses in breathing (called apnea), or instances of abnormally shallow breathing or low respiratory rate (called hypopnea), that occurs mostly during sleep. Apnea is the medical term for the suspension of external breathing for greater than 10 seconds, during which time there is no movement of the muscles of respiration. Hypopnea is considered clinically significant if there is a 30% or more reduction in respiratory flow lasting for 10 seconds or longer and an associated 4% or more de-saturation in the person's blood oxygen level.

Sleep Apnea is further classified as Central Sleep Apnea (CSA), Obstructive Sleep Apnea (OSA), and complex or mixed (a combination of CSA and OSA). Obstructive Sleep Apnea is the largest sleep disorder, constituting more than 80% of all sleep apnea cases. According to the World Health Organization, approximately 100 million known cases of people worldwide suffer from OSA. In the United States alone, OSA is estimated to affect approximately 22 million working adults; out of that, about 4% of men and 2% of women have symptomatic moderate or severe OSA, affecting approximately 1.3 million adults. OSA is widespread with similar prevalence estimates from Europe, Australia and Asia. It is estimated that less than 25% of OSA sufferers worldwide have been diagnosed. OSA sufferers who generally sleep alone are often unaware of the condition, without a regular bed-partner to notice and make them aware of their symptoms. In general, men are twice as likely to have OSA as women. Obesity also plays an important role in OSA, as more than half of people with OSA are overweight. OSA, a relatively new market in the medical field, is gaining momentum at a fast pace, especially in developed geographies.

In CSA, breathing is interrupted by a lack of respiratory effort usually due to instability in the body's feedback mechanisms that control respiration (involving the brain, blood oxygen level, phrenic nerve, and the diaphragm). Whereas in OSA, breathing is repeatedly interrupted by a physical block to airflow or narrowing in the upper pharynx in spite of respiratory effort, and snoring is common. Although a very minor degree of OSA is considered to be within the bounds of normal sleep, many individuals experience episodes of OSA at some point in life, and a small percentage of people are afflicted with chronic, severe OSA (5 to more than 30 episodes per hour).

In people with OSA, pharyngeal or windpipe muscles (which are normally active or have the muscle tone while awake) relax or lose the muscle tone during sleep, and especially during the REM (Rapid-Eye-Movement) sleep and gradually allow the pharynx to collapse. However, an abnormal airway is present in OSA patients even when awake. The level of pharyngeal collapse varies between patients, but most often occurs at the velopharynx, retroglossal and base-of-tongue level. The tongue is a frequent cause of upper airway blockage in obstructive sleep apnea, as it can collapse toward the back of the throat during sleep, therefore contributing to obstruction of the airway. Collapse of the pharyngeal airway can block airflow or significantly restrict airflow, both of which can cause blood oxygen reduction or de-saturation. Large tonsils or adenoids or other anatomical structures such as a deviated septum, enlarged tongue, or receding chin can cause a narrowed airway. Obesity may directly result in airway obstruction because of fat deposition around the airway or more commonly from thickening of the pharyngeal musculature and supporting tissues. OSA patients with even normal BMI (body mass index) frequently have facial and mandible abnormalities that predispose to upper airway obstruction. OSA occurs more often in those who have a consistent nasal obstruction. A person with family history of sleep apnea may be at an increased risk. Use of alcohol, sedatives or tranquilizer substances also relaxes the muscles in the throat. Smokers have a higher chance of OSA. Medical statistical data shows that in the USA, 50% of 21 million American patients with Type II diabetes have OSA; 40% of 44.7 million with hypertension and 68% of 5 million with CHF (heart failure) also have OSA.

Episode of OSA is terminated by a brief arousal or a lighter stage of sleep, accompanied often times by sweat and ultimately activation of the upper airway dilator muscles and restoration of airway patency (opening). This fragmented and tortured sleep cycle occurs repeatedly throughout the night commonly resulting in daytime hyper-somnolence or sleepiness; the consequences are excessive fatigue, headache, difficulty concentrating, higher likelihood of auto accidents, irritability, gastro-esophageal reflux symptoms (due to the negative intra-thoracic pressure during attempted inspiration during upper airway obstruction), loss of productivity, and long-term metabolic and central nervous system abnormalities and associated increased risk (by 1.5 to 4 times) of co-morbidities such as hypertension, stroke, cardiac ischemia, arrhythmia and sudden death. Healthcare studies on OSA have found that people with untreated OSA cause twice as higher healthcare costs than similar cases without OSA.

OSA is most generally diagnosed with polysomnography, a gold standard sleep test. This overnight test correlates oxygen saturation, EEG, abnormal sleep associated movements with apnea and hypopnea episodes. When the findings are subtle, an advanced image processing is used to facilitate diagnosis. Imaging is used primarily for treatment planning of patients with OSA and not for primary diagnosis.

Therapies for OSA that are currently practiced are: Continuous positive airway pressure (CPAP), mandible advancement surgery, septoplasty, palatoplasty, uvuloplasty and oral dental appliances. Electrical stimulation therapy for pharyngeal nerves and/or muscles is in the pipeline.

CPAP therapy is currently the most effective or a gold standard treatment for OSA. A nasal and/or full face mask, a hose and an air compressor or blower by the bedside are used to pressurize the airway to help stent the airway open during sleep. CPAP is shown to be very effective in treating OSA and reducing the day-time symptoms of OSA when used and adjusted correctly. Unfortunately only about one-third of the patients seem to tolerate the CPAP devices and it has been reported that less than half of the patients follow their sleep consultant's prescriptions for sleep therapy and use the device. The commonest causes of CPAP intolerance are claustrophobia, discomfort, nasal obstruction and retroglossal narrowing.

Treatment by surgery is considered for some patients who are not able to tolerate other forms of treatment and/or have a significant anatomical abnormality that is the cause for their condition. The tongue collapsing in some cases is resolved with genioglossus advancement. The genioglossus is the primary muscle of the tongue and is attached to a small bony projection on the interior of the lower jaw. During genioglossus advancement surgery, this small projection is moved forward and the tongue attachment is repositioned to the anterior so that it is less likely to collapse to posterior position and block the airway during sleep. This procedure is performed in a hospital surgery center under general anesthesia and takes approximately 30 minutes. While speech and swallowing may not be affected, the procedure is typically associated with pain, swelling and occasional minor numbness of the lower front teeth. In some cases, Uvulopalatal flap and radiofrequency reduction surgery is performed to the back-of-tongue in order to maximize airway improvement. Clinical success rates with surgery are mixed because the exact mechanical problem of obstruction is unclear or ambiguous, extent of surgery may be limited for practical reasons and the soft tissue itself may re-grow. The surgery may cause scar tissue and there is the probability of infection and small percentage of mortality associated with any surgery.

Oral or dental appliances are recommended mostly for individuals with mild to moderate OSA. Oral appliances are designed to keep the airway open by advancing the lower jaw or tongue forward during sleep. In general, oral appliances are not considered as effective as CPAP or surgeries in treating OSA and some patients have difficulty wearing one through the night as it exerts constant pressure on the mouth anatomy. There are other difficult treatments for OSA that include behavioral control of sleep posture, tongue training etc. Due to lack of patient compliance and difficulty in usage, the most effective therapy of all current therapies, namely CPAP, is not the ideal solution.

3 startup medical device companies in the USA are currently at different stages of development and approval process of an implantable electrical stimulation therapy for OSA. This technology involves implanting an electrical stimulation device inside the body or under the skin in the pectoral chest region (just like a cardiac Pacemaker) and running single or multiple stimulation leads subcutaneously to the branched site of hypoglossal nerve under the chin. Hypoglossal nerve is the 12th cranial nerve (XII) emerging from the medulla oblongata, leading to the tongue. Involuntary activities such as swallowing to clear mouth of saliva are controlled by the hypoglossal nerve; however, most functions are voluntary. The electrodes implanted at the hypoglossal nerve stimulate wholly or selectively the motor fibers of the nerve to contract and restore the dormant muscle tone to the genioglossus and other tongue muscles involved in the pharyngeal airway during sleep, so the tongue is protruded away from the pharyngeal airway. These implanted devices either use an open loop system (i.e. active stimulation without regard to the inspiration/expiration cycle) or a closed loop system (i.e. synchronize the stimulation to the inspiration cycle which is sensed by an appropriate sensor subcutaneously implanted in the chest/diaphragm region). The patient's physician determines and presets the stimulation energy in the implanted device and the patient thereafter simply turns the therapy ON at sleep time and OFF after sleep. Though this implanted therapy system eliminates patient compliance and executes the therapy automatically, it is an invasive, complex and expensive procedure. There could be complications with implanting the electrodes at the hypoglossal nerve, a potential for hypoglossal nerve damage or internal carotid artery and jugular vein puncture, and potential pain, swelling and unintended numbness or paralysis in regions of mouth and teeth, not to speak of infections and mortality associated with any surgery. Some lateral branches of the hypoglossal nerve innervate certain portions of tongue muscle which when stimulated by electrodes in wrong location may actually retract rather than protrude the tongue thereby blocking the airway. A simple, non-invasive electrical stimulation method is a better solution.

### BRIEF SUMMARY OF THE INVENTION

In contrast to all of the above cumbersome, difficult-to-comply, invasive, risky and expensive methods of treatment for OSA, the present invention illustrates the use of a simple non-invasive (i.e. no incision, no implantation), easily removable retainer or denture-like intraoral electrical Stimulator or Pacemaker-like device assembly for the treatment of OSA. In a general embodiment, the device is installed easily on the lower teeth inside the adult or young adult patient mouth by the patient him/herself, and bilaterally (i.e. across left and right sides) or unilaterally (one or both sides, but not across sides) stimulates the posterior to mid portion of the extrinsic tongue protruder muscles, namely the genioglossus muscle and the back-of-tongue intrinsic muscles, where these muscles are implicated in OSA for the collapse of the Oropharyngeal airway (oral part of the pharynx which reaches from the Uvula to the level of the Hyoid bone) at the retroglossal and base-of-tongue level. Thus the key aspect of the present invention is that obstructive sleep apnea is treated by electrically stimulating certain muscles of the Oropharynx in order to contract and thereby pull open or widen the obstructed airway. The device in this invention may function in an open loop configuration (i.e. continuously stimulating the muscles during sleep, without regard to intrinsic respiration cycle); or in a closed loop configuration (i.e. stimulation synchronized to intrinsic respiration cycle and/or to the apnea episodes, based on the detected electromyographic (EMG) activity or other movements of the genioglossus muscle and tongue muscles).

More than a decade ago, Schwartz et al. studied and assessed by means of the upper airway critical pressure (Pcrit), that electrical stimulation of tongue protruder muscles, the genioglossus muscle increased airflow in the OSA patients; they also determined that stimulation of the tongue refractor muscles, the styloglossus muscles however decreased airflow. The position of the tongue is considered to be determined by the balance of contraction force between the tongue protruder and the retractor muscles. In 2001, Oliven et al. also studied and found that the electrical stimulation of the posterior region of the tongue muscles in particular as well as the anterior region was more effective in improving the pharyngeal patency during sleep than stimulation of the anterior region alone, suggesting that it is the preferential activation of the genioglossus muscle that produced the most protrusion of the tongue. Bishara et al. also demonstrated that selective upper airway dilatory muscle (direct) stimulation in spontaneously breathing anaesthetized dogs reduces airway resistance in the presence of airway obstruction and releases airway occlusion, with the genioglossus being the most effective muscle. In brief, contraction of the tongue musculature induced by electrical stimulation stiffens the Oropharyngeal airway wall by contracting the attached tongue muscles and improves the airway patency. Mezzanotte et al. (1992) observed that OSA patients have significantly greater genioglossal electromygraphic activity compared to control or non-OSA group of patients during wakefulness and this neuromuscular compensation present during wakefulness in apnea patients may be lost during sleep leading to airway collapse. This reduction or loss in EMG activity of genioglossus muscle during sleep may be detected and used for closed loop activation of electrical stimulation.

Present invention relates to a removable intraoral Stimulator system and stimulation method for the treatment of OSA. Treatment may be therapeutic, prophylactic or preventative as determined by the patient's physician. The basic or standard embodiment consists of a rechargeable battery-powered electrical Stimulator device (a.k.a. Pacemaker or pulse generator), with one or more micro-controllers and several electronic sub-systems for the generation and control of stimulation pulses for the excitation and contraction of tongue protruder muscles, primarily the genioglossus and other appropriate tongue muscles, preferentially in the posterior or base-of-tongue region. The electrical Stimulator device assembly is enclosed in a hermetically sealed encapsulation or housing of any biocompatible material such as titanium, stainless steel, epoxy, silicone, polyurethane, thermoplastic, or thermosetting polymers. The encapsulation is finish-coated in a soft non-abrasive dental or other biocompatible polymer material to be comfortable in the mouth. The Stimulator's electrode configuration typically consists of two pairs of electrodes that are placed ventral-laterally at the posterior to mid genioglossus muscle surface region or base-of-tongue for bilateral stimulation of the above mentioned tongue muscles and also for acquiring the electromyographic (EMG) signal of the genioglossus muscle; the electrode configuration may also consist of a single pair of electrodes on the either or both sides for unilateral stimulation. The smoothly shaped electrodes do not make any incision into the muscle but contact the sublingual muscle surrounded by saliva. The electrical Stimulator device assembly and the electrodes are connected together by insulated water-tight leads. In the case of bilateral stimulation, the Stimulator device and the bilateral pairs of electrodes are integrally embedded into or attached to a removable dental wire-frame or dental oral housing appliance covering full or partial length of the lower teeth; in the case of unilateral stimulation, the Stimulator device and a single pair of electrodes are integrated into a molar teeth clip that attaches to the molars. Inductive half-duplex (bidirectional, non-simultaneous) data communication telemetry sub-system is used for setting up or programming the stimulation and other system parameters in the Stimulator device and for receiving system information back from the Stimulator device to its external sub-systems. An external inductive Recharger sub-system, a mains operated appliance with a rechargeable battery backup is used for recharging the rechargeable battery in the intraoral Stimulator device, by transferring charge energy or power through electromagnetic induction. Another external inductive Programmer sub-system, a non-rechargeable battery operated hand-held appliance is used by the patient's sleep medicine physician to program the stimulation and system parameters in the intraoral Stimulator device, through electromagnetic inductive transmission protocol.

The removable dental oral housing and molar teeth clip are mostly a custom-made plastic molded parts fabricated utilizing a replicate model of lower teeth structure by the conventional dental impression methods, and a thermal mold forming process using conventional dental materials and other biocompatible plastic materials. The oral housing and molar teeth clip appliance simply serves as an integral holder for the Stimulator device, electrodes and leads; it can engage all lower teeth or only some lower teeth consisting of at least one molar tooth and/or the gingival tissue surrounding the molars and one or more other teeth particularly the incisor teeth on both sides of the mouth, and is constructed for easy installation into and removal from the mouth by the OSA patient him/herself. The electrical Stimulator device has appropriate muscle stimulation circuits known to those in the art of human smooth and skeletal muscle and nerve stimulation, a compact rechargeable battery and an electromagnetic inductive receiver/transmitter and battery charger circuit, and bidirectional or half-duplex data communication circuit, all packaged in a hermetically sealed biocompatible encapsulation or housing and integrally attached to or embedded in the dental oral housing or molar teeth clip. The intraoral removal Stimulator device assembly in the dental housing can be cleaned with medi-wipes.

### BRIEF DESCRIPTION OF THE DRAWINGS

The purpose of the drawings in this invention is to illustrate the exemplary embodiments and visually show the aspects, features and advantages of the invention as explained above and in the following detailed description and claim sections. The drawings are not to scale and only meant to convey the idea of invention.

FIG. 1—Annotated anatomical illustration of the Human Mouth and Pharyngeal Airway (file image copy from Grays Anatomy, Gray994.png), illustrates the various muscles of the mouth and pharynx.

FIG. 2—Muscle anatomy of Tongue, illustrates the various intrinsic and extrinsic muscles of the tongue.

FIG. 3—Regions of Bilateral (side-to-side) and Unilateral (one side) Stimulation of Genioglossus Muscle in adult oral cavity (underside of the Tongue, proximal to Molars).

FIG. 4—Components of the complete Intraoral Stimulator System, consisting of an Intraoral Stimulator device assembly, an external inductive Recharger appliance, and an external inductive Programmer appliance.

FIG. 5—Perspective View of the complete Intraoral Stimulator System, shows the Intraoral Stimulator device assembly in a dental oral housing laid in the cradle of an external Recharger appliance, and an external Programmer appliance for programing the Stimulator device.

FIG. 6A—Assembly concept of Intraoral Stimulator device and Bilateral Electrodes in a dental wire-frame, shows the Stimulator device and electrodes assembled in a hollow wire-frame, for stimulating sublingual muscle bilaterally.

FIG. 6B—Assembly concept of Intraoral Stimulator device and Bilateral Electrodes embedded into a mouth-guard-like plastic dental oral housing, shows the Stimulator device and electrodes assembled into a dental oral housing, for stimulating sublingual muscle bilaterally.

FIG. 6C—Assembly concept of Intraoral Stimulator device and Unilateral Electrodes embedded into a dental molar teeth clip, shows Stimulator device and electrodes, for stimulating sublingual muscle unilaterally.

FIG. 6D—3-dimensional concept view of the Intraoral Stimulator device and Bilateral Electrodes in a mouth-guard-like dental oral housing.

FIG. 7A—Intraoral Stimulator device in a dental oral housing for bilateral stimulation, shown in Left-side or Right-side orientation and placed outside the teeth between teeth and cheek.

FIG. 7B—Intraoral Stimulator device in a dental oral housing for bilateral stimulation, shown in Left-side or Right-side orientation and placed inside the teeth between tongue and palette space.

FIG. 7C—Intraoral Stimulator device and separate Tx/Rx Coil assembly in a dental oral housing for bilateral stimulation, shown in Left-side or Right-side orientation and placed outside the teeth between teeth and cheek.

FIG. 8—Intraoral Stimulator device in a dental molar teeth clip for unilateral or single-sided stimulation, shown in Left-side or Right-side orientation and placed outside the teeth between teeth and cheek.

FIG. 9—Functional Block Diagram of the electrical intraoral Stimulator device, shows the micro-controllers, all electronic sub-systems, sensors, battery, and electrode interface.

FIG. 10—Electrode Configuration for a Bilateral Single-Channel Stimulation system with two pairs of electrodes, shows pairing of any combination of right/left bilateral electrodes R1/R2 and L1/L2 to PACE+ & PACE−, the single channel biphasic stimulation signals.

FIG. 11—Electrode Configuration for a Bilateral Dual-Channel Stimulation system with two pairs of electrodes, shows pairing of any combination of right/left bilateral electrodes R1/R2 and L1/L2 to PACE1+/PACE1− and PACE2+/PACE2−, the two channels of biphasic stimulation signals.

FIG. 12—Functional Block Diagram of (inductive) External Recharger Appliance, shows the micro-controller and all electronic sub-systems including battery.

FIG. 13—Functional Block Diagram of (inductive) External Programmer Appliance, shows the micro-controller and all electronic sub-systems including battery.

### DETAILED DESCRIPTION OF THE INVENTION

Description in this section explains the general principles and details of invention. The scope of the invention itself shall be determined by the claims section of the invention.

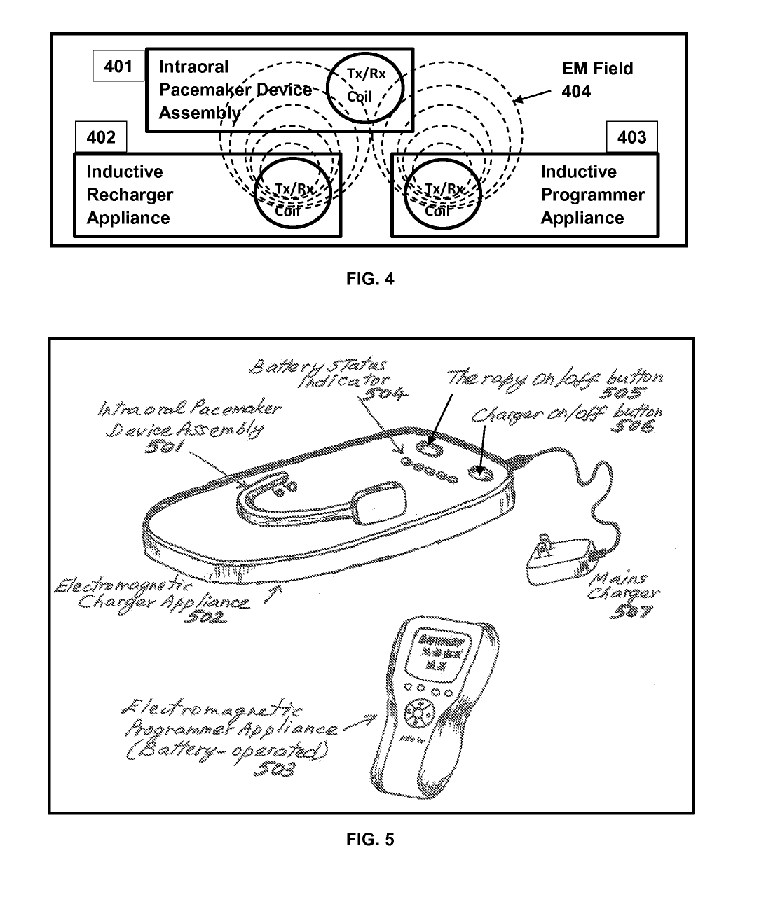

As explained in the summary section, the present invention relates to a non-invasive, removable intraoral Stimulator device, system and muscle stimulation method for the treatment of obstructive sleep apnea (OSA) disorder in human adults and young adults. The invention is an electrical Stimulator device that's powered by a rechargeable battery. The complete intraoral Stimulator system is conceptually illustrated in FIG. 4. The rechargeable battery inside the Stimulator device assembly 401 is recharged inductively by the Recharger Appliance 402, and the Stimulator device is programmed inductively by the Programmer Appliance 403 through the electromagnetic (EM) field 404. The perspective view or concept of the complete Intraoral Stimulator System is illustrated in FIG. 5; the Intraoral Stimulator device assembly in a dental oral housing 501 is laid in the cradle of an external Recharger appliance 502 for recharging the Stimulator device battery, and an external Programmer appliance 503 is used for programming the Stimulator device. The Recharger appliance 502 is ac mains operated with a medical grade isolation adapter 507, and has buttons for Charger On/Off 506, Therapy On/Off 505, and (Stimulator device) Battery Status indicator 504.

Structural and functional details of the Intraoral Stimulator system are as follows:
Electronic hardware, sensors, magnetic transmit/receive (Tx/Rx) coil and battery are packaged snugly and safely in a hermetically sealed biocompatible encapsulation or housing, and coated with a soft non-abrasive dental or other biocompatible polymer material to feel smooth and comfortable in the mouth.
The invention Stimulator device has typically two pairs of bilateral electrodes (or a single pair of unilateral electrodes), of biocompatible materials known in the art of implantable biomedical electrical stimulation and bio-signal acquisition, and is located ventral-laterally and sublingually at the posterior to middle section under the tongue for recruiting a large section of the genioglossus muscle and base-of-tongue for stimulation to regain muscle tone during sleep.
The encapsulated Stimulator device and the insulated stimulation electrodes are connected and embedded into a hollow dental retainer wire-frame or a mouth-guard like dental oral housing (in the case of bilateral configuration) or a molar teeth clip (in the case of unilateral configuration) on the lower teeth to make up what is called “Intraoral Stimulator Device Assembly”.

Basic or standard embodiment of the Stimulator device operates in an open loop configuration, where the stimulation therapy is executed without regard to the intrinsic respiration cycle or patient activity.

Advanced or different embodiments of the Stimulator device incorporate sensors:
An Accelerometer sensor for determining the patient's position and activity and using that information for qualifying or regulating the stimulation therapy,
A Temperature sensor for determining if the device is inside the mouth or outside and using that information for controlling power On/Off in the device,
A Piezoelectric film sensor for possibly detecting the intrinsic respiratory cycle by sensing rhythmic moves in the mouth cavity and using that data for regulating the stimulation therapy,
An EMG acquisition and detection sub-system using one of the pairs of electrodes, for detecting the increase or decrease in genioglossus muscle activity and qualifying the muscle stimulation therapy on that basis. The EMG activity level during sleep or apnea episode is suppressed or much lower (when the genioglossus muscle tone is suppressed during sleep in OSA patients) than its baseline level during the patient's wakeful state, and the EMG activity goes back to the baseline level in wakeful state; such information may be used for turning On or Off the stimulation therapy.

In the advanced embodiments of the Stimulator device, one or more sensor information as above is used for the closed loop control of the Stimulator device, namely onset/offset or synchronization of the stimulation therapy to the patient position, activity, respiration cycle and EMG activity increase or decrease.

Rechargeable battery in the Stimulator device is charged inductively (i.e. by electromagnetic field) by an external Recharger appliance, periodically or once in a few days, by removing the intraoral Stimulator device assembly from the mouth and placing it in the cradle of the Recharger appliance as shown in FIG. 5. The Stimulator device incorporates a Tx/Rx coil for inductive coupling with the Recharger appliance. The Recharger appliance is as well used by the patient for turning On/Off the intraoral Stimulator device and for viewing the internal battery status of the device.

At the time of oral implant and at subsequent follow up visits, the patient's physician programs the patient's intraoral Stimulator device with appropriate stimulation therapy parameters (such as stimulation pulse strength or amplitude, pulse width, pulse repetition frequency, and operating mode) using an external hand-held Programmer appliance which works on electromagnetic inductive telemetry schema like the Recharger appliance. The data communication is bidirectional half-duplex, whereby the physician is also able to download and view the stimulation data and system information from the intraoral Stimulator device on the Programmer.

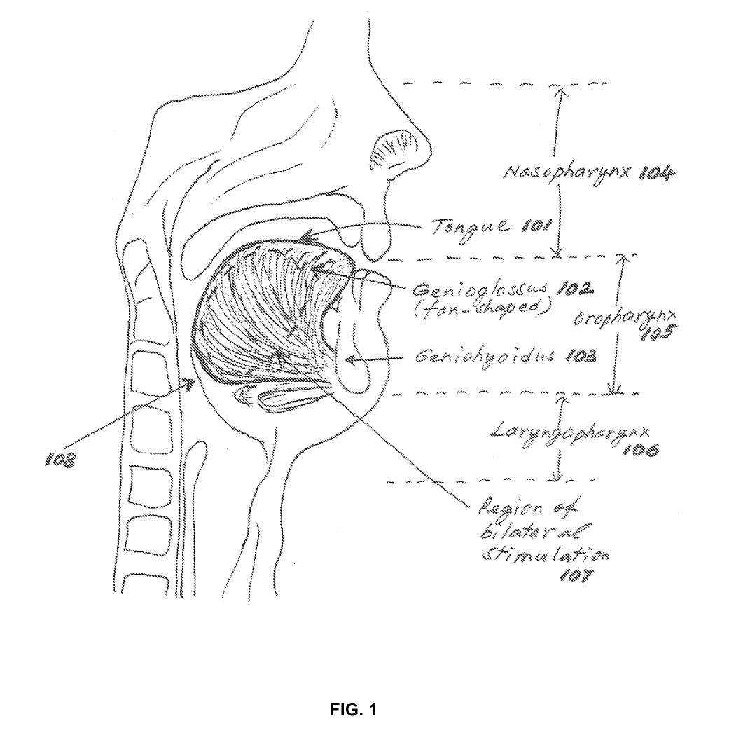

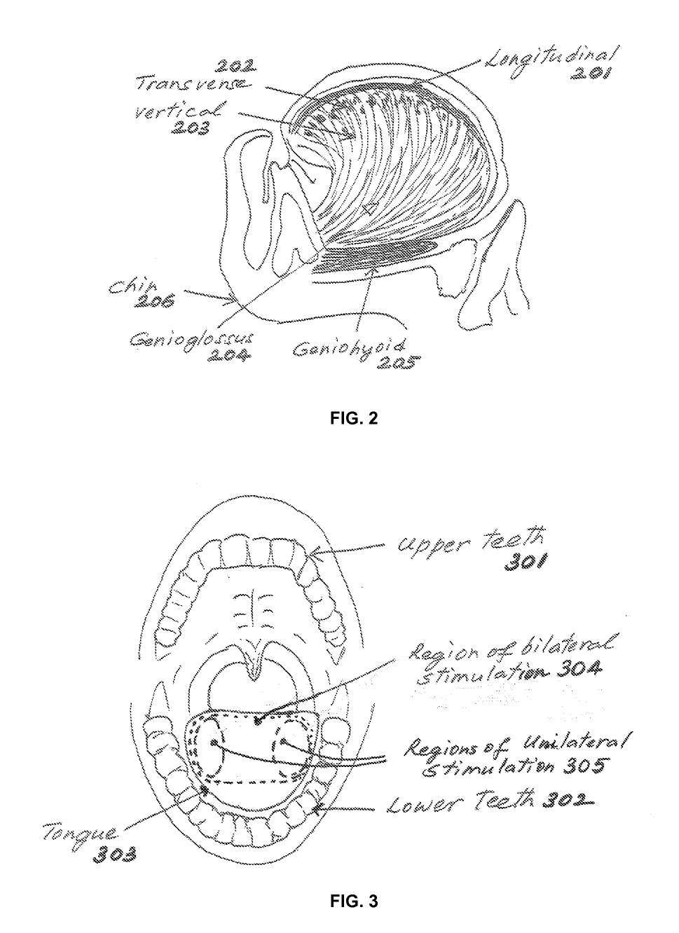

The muscle of interest for stimulation in this invention is primarily the genioglossus muscle in the adult or young adult mouth, a tongue protruder muscle; and the region of interest for stimulation is the posterior to mid region of the genioglossus or base-of-tongue muscle, ventral-lateral region of the tongue as illustrated in the FIGS. 1, 2 and 3. As shown in FIG. 1, the oral part of the pharynx airway 108 is blocked by the Tongue muscle 101 by relaxing and falling back into the airway during sleep due to loss of tone. Stimulation of the genioglossus muscle 102, preferentially in the posterior region 107 regains the muscle tone. As shown in FIG. 2, the fan shaped genioglossus muscle 204 is the muscle of interest for stimulation. FIG. 3 shows the intended regions of bilateral stimulation 304 and unilateral stimulation 305 under the tongue, the ventral-lateral sublingual or genioglossus muscle 303.

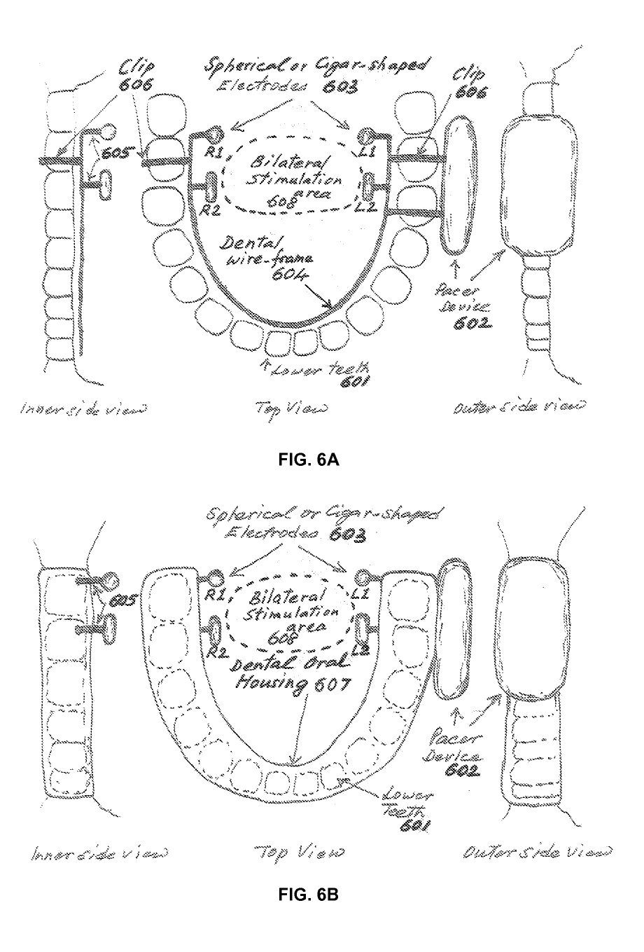

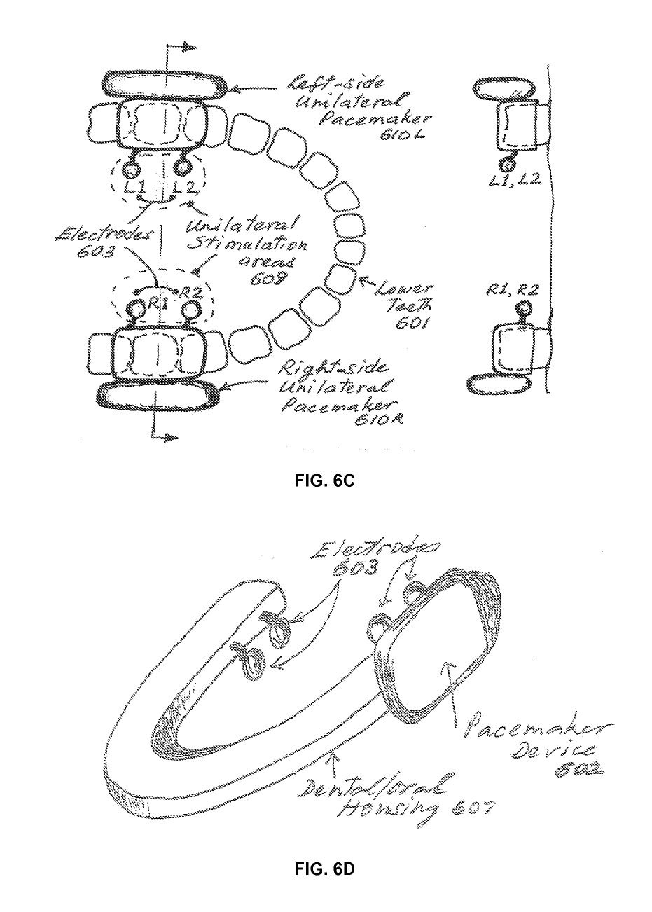

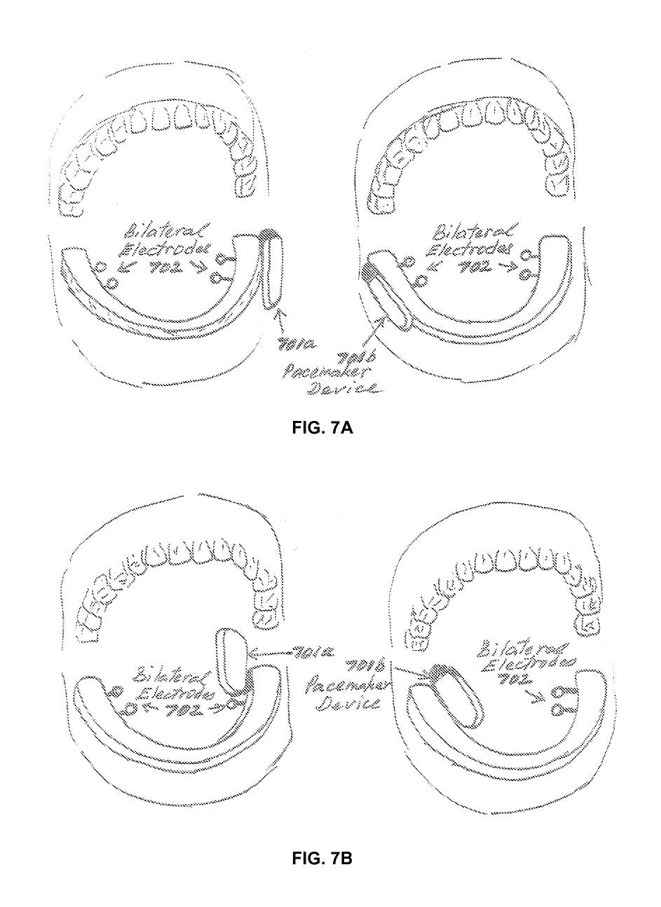

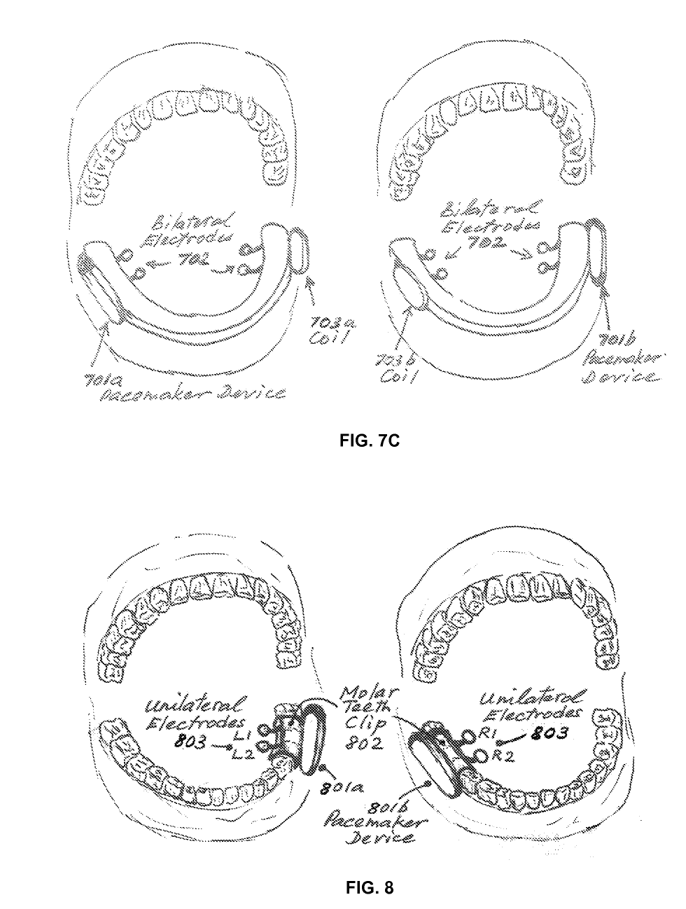

As shown in FIGS. 6A, 6B, the two bilateral pairs of electrodes 603 (R1/R2 and L1/L2), made of Silver-Silver Chloride (Ag—AgCl) or Platinum-Iridium (Pt—Ir 90/10) or gold plated silver material, are attached through insulated lead wires and a water-tight sheath of soft and flexible silicone material 605 to the Stimulator device encapsulation 602. The dental wire-frame 604 in FIG. 6A attaches to the molars or near molars on both sides of the lower teeth 601 with plastic clips 606, and electrode wires run from one side to the other side through the hollow wire-frame. The dental oral housing 607 in FIG. 6B sits over the lower teeth 601. In the unilateral stimulation configuration, as shown in FIG. 6C, the Stimulator device and a single pair of unilateral electrodes 603 (R1/R2 or L1/L2) are connected and attached through a dental molar teeth clip; there can be only one device (on one side) or two devices (on both sides) that operate completely independently and asynchronously of each other (synchronization is not needed as the intent is simply to generate and maintain muscle tone in the genioglossus muscle). The electrodes in bilateral and unilateral stimulation configurations (603 in FIGS. 6A, 6B, 6C) are designed in such a way as to make good contact with the genioglossus muscle underside of the tongue. Electrodes are either spherical (of 2-10 mm diameter), or pancake shaped with curved top and bottom surfaces (of 8-40 mm circumference and 2-10 mm thickness), or short cigar shaped with smooth hemispherical ends (of 2-10 mm diameter & 5-10 mm length), and surface-textured to minimize polarization effect and produce uniform current density; the electrodes are placed in such a way that they can contact the genioglossus muscle uniformly, the idea being to avoid “hot-spot” current densities, increase the surface contact area or reduce the electrode impedance thus resulting in higher signal-to-noise ratio, low exogenous noise signal pickup (which is particularly useful for EMG pickup), and greater stimulation efficiency. FIG. 6D shows a 3-dimensional concept view of the intraoral Stimulator device 602 and two pairs of bilateral Electrodes 603 embedded in a dental oral housing 607 that sits over the lower teeth. The Stimulator device encapsulation attachment to the dental oral housing can be left or right-side oriented and placed outside the teeth between the teeth and cheek, as shown as 701a and 701b in FIG. 7A. The Stimulator device encapsulation attachment to the dental oral housing can instead be placed inside the teeth between the tongue and palette, as shown as 701a and 701b in FIG. 7B. In case it is not possible to encapsulate all components into a single encapsulation housing, then the Tx/Rx coil can be separated from the main Stimulator device encapsulation housing and encapsulated into a separate housing and attached to the dental oral housing at opposite side to the main Stimulator housing, as shown as 703a and 703b in FIG. 7C. The electrodes 702 in FIG. 7A, FIG. 7B & FIG. 7C are always inside the teeth under the tongue. As shown in FIG. 8, the Intraoral Stimulator Device 801a or 801b and a single pair of unilateral Electrodes 803 embedded in a dental molar teeth clip 802 can be left or right-side oriented and placed outside the teeth between the teeth and cheek; the electrodes 803 are always inside the teeth under the tongue. FIG. 8 shows a single device on only one side, but two such devices can be placed on both sides, but they operate independently.

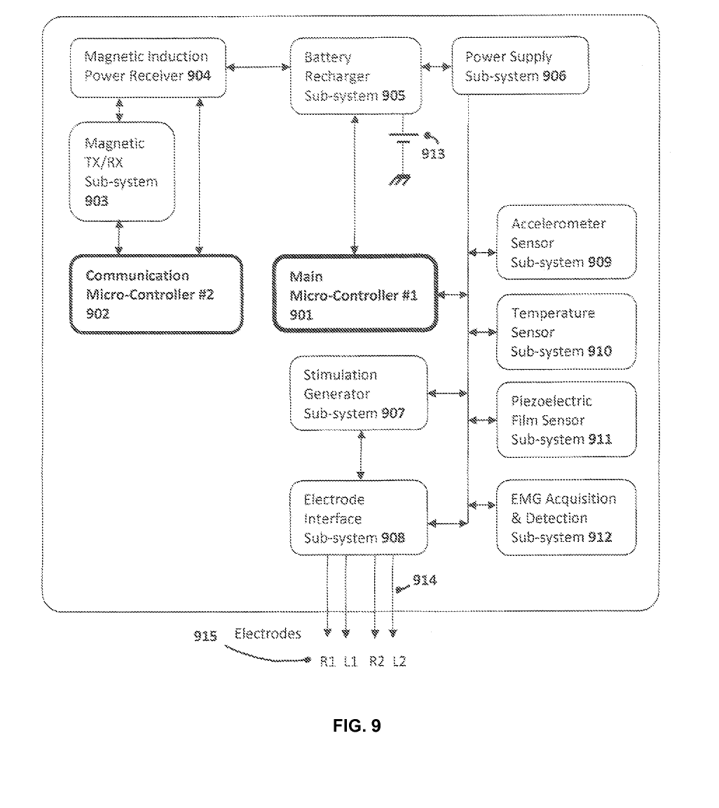

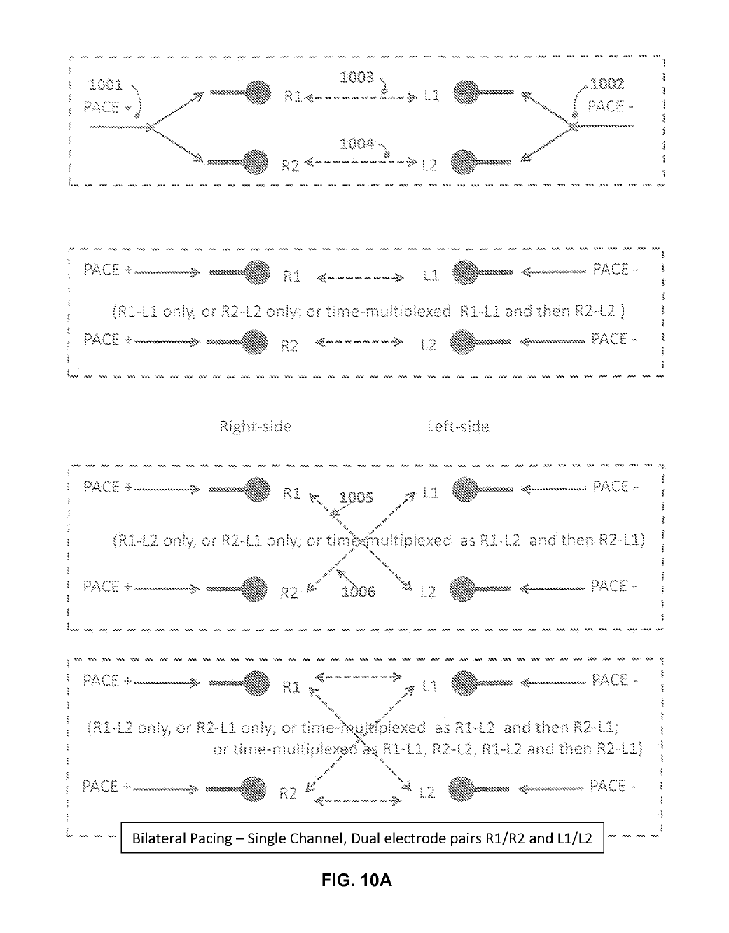

As shown in FIG. 9, the Stimulator device consists of one or two micro-controllers for separate functions; a Main Micro-Controller 901 for controlling most sub-systems, and another (optional) Communication Micro-Controller 902 for managing the inductive bidirectional power and telemetry data communication schema. In the absence of the optional communication controller, the main micro-controller manages the communication sub-system as well. The intelligence of the Stimulator device lies in the firmware executing in the micro-controllers, as the system is software (firmware) controlled hardware system. As shown in FIG. 9, functional sub-systems 901-912 of the Stimulator device, easily recognizable by someone familiar in the art of implantable electrical stimulation of muscle/nerve, are as follows: Inductive Transmit/Receiver Tx/Rx sub-system 903 (a tuned inductor and capacitor, switched magnetic transmitter, receiver and detector system), Magnetic Induction Power Receiver sub-system 904 (a voltage rectifier, converter and capacitive storage system), Battery Recharger sub-system 905 (Li-ion battery charger, charge limiter and discharge controller system), Power-Supply sub-system 906 (a complete multiple voltage regulator system), Stimulation Generator sub-system 907 (an independent dual channel constant current pulse generator system with programmatic control of pulse parameters), Electrode Interface sub-system 908 (a programmatic interface control between the dual channel pulse generator outputs and two pairs of pacing electrodes) and optional sensor sub-systems (Accelerometer sub-system 909, Temperature Sensor sub-system 910, Piezoelectric Film Sensor sub-system 911 and EMG Acquisition & Detection sub-system 912). A rechargeable battery 913 of popular Li-ion chemistry of suitable geometry and capacity to provide full function pacing for 2 or 3 days with a single charge and which can be recharged and maintained by the Battery Recharger sub-system 905. All sub-systems including the micro-controllers may contain custom integrated circuits and/or off-the-shelf integrated circuits.

In FIG. 9, the Magnetic Tx/Rx sub-system 903 consists of a single or separate Transmit (Tx) and Receive (Rx) electrical coils that are tuned or resonant to the inductively coupled transmitted energy from the external Recharger appliance, and a half-duplex inductive communication receiver and transmitter circuits. The Magnetic Induction Power Receiver sub-system 904 converts the (inductively coupled) received electromagnetic energy through a rectifier converter system to a voltage. The Battery Recharger sub-system 905 is responsible for using the output of the received voltage from the Magnetic Induction Power Receiver sub-system 903 and charging and discharge-monitoring of the Li-ion battery, so the battery does not discharge below a safe operating level. Other operating voltages as required by the Stimulator system are generated by the Power Supply sub-system 906. The bi-directional telemetry data communication protocol including checksum and error detection is handled by the communication micro-controller or the main micro-controller in the case of a single controller system.

In FIG. 9, the Stimulation Generator sub-system 907, under the DMA (direct memory control) software control of the main micro-controller generates up to two independent channels of symmetrical biphasic stimulation pulses of current amplitude of 0 to 15 mA over a tissue load range of 200 to 2500 Ohms, pulse width of 100 to 1000 uSec, pulse repetition rate of 5 to 150 pulses per second (pps) and 0 to 500 mSec inter-channel delay. The generator can be programmed to generate these stimulation pulses normally at a fixed pulse amplitude, pulse width and pulse repetition rate; or it can modulate or vary any one or more of these parameters automatically in a programmed pre-defined cyclical pattern, including initial gradual step-up in pulse amplitude. Whether a fixed or modulation pattern, the generator can be programmed to generate the pattern either continuously for a programmed time duration of 1 minute to 12 hours; or cyclically burst or turn On the pattern for a time duration of 1 second to 2 minutes and turn Off the pattern for a time duration of 1 second to 2 minutes, and repeat the cycle continuously or for a time duration of 1 minute to 12 hours. The stimulation current pulses are generated by a closed loop voltage regulator system that continuously monitors the current through the load and automatically adjusts the voltage source so a constant current is delivered to the load.

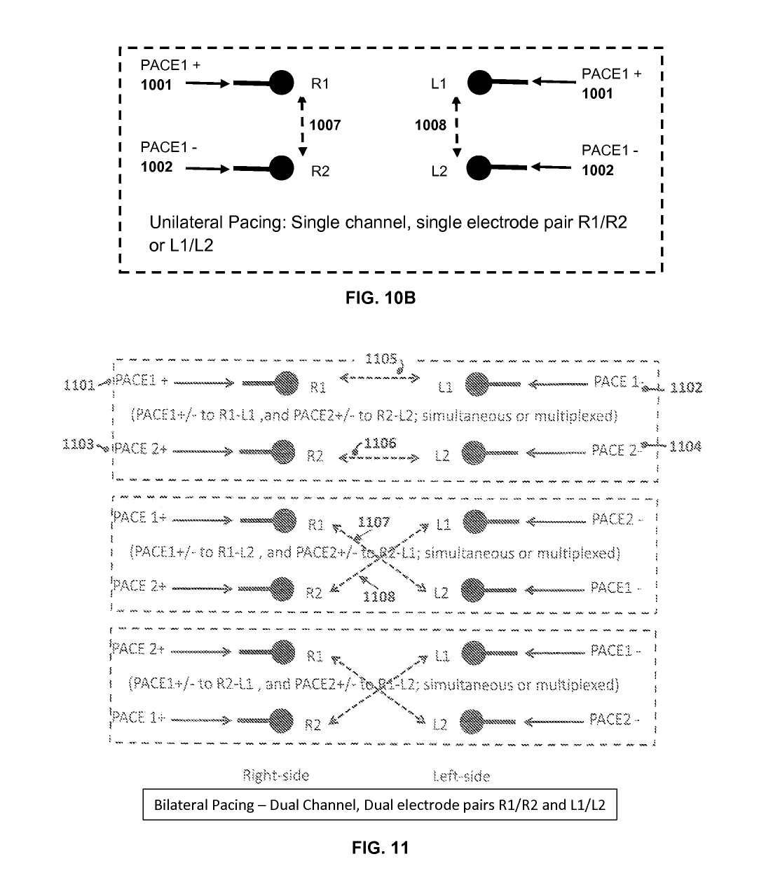

In the same FIG. 9, the outputs of one or two stimulation channels are routed to the two pairs of electrodes (R1/R2 and L1/L2) 915 (corresponding to the bilateral electrodes 603 shown in FIGS. 6A, 6B, 6C), through the physical connection interface 914, by appropriate electronic switches at appropriate times during the stimulation protocol, by the Electrode Interface sub-system 908. In an embodiment using a single stimulation channel for bilateral stimulation, the stimulation output signal pair PACE+ 1001 & PACE− 1002 as shown in FIG. 10A can be time-multiplexed and connected to one or more of the programmed pairs of electrodes R1-L1 1003, R2-L2 1004, R1-L2 1005 or R2-L1 1006, by the Electrode Interface sub-system 908 in FIG. 9. This is done in order to possibly recruit a larger region of the genioglossus muscle and/or to change the axis or orientation of the stimulation current vector in the genioglossus muscle structure, hopefully for the most effective protrusion of the tongue away from the Oropharyngeal airway. In an embodiment using a single stimulation channel for unilateral or one-sided stimulation, the stimulation output signal pair PACE+ 1001 & PACE− 1002 as shown in FIG. 10B can be connected to the single pair of electrodes R1-R2 1007 or L1-L2 1008, by the Electrode Interface sub-system 908 in FIG. 9. In another embodiment using two stimulation channels for bilateral stimulation, the two channels of stimulation output signals PACE1+ 1101 & PACE1− 1102 and PACE2+ 1103 & PACE1− 1104 as shown in FIG. 11 can be connected to the pairs of electrodes R1 -L1 1105, R2-L2 1106, R1-L2 1107 or R2-L1 1108 as shown in the same figure, either simultaneously or in a time-multiplexed manner (with delay) by the Electrode Interface sub-system 908 in FIG. 9. Again, this is done in order to simultaneously recruit a larger region of the genioglossus muscle. Another possible use for the simultaneous or multiplexed stimulation in different electrode pairs may be to put the underlying pain nerves into depolarization or refractory state while the muscle is being recruited for contraction in the other electrode pair.

The stimulation parameters and the stimulation protocol (namely fixed or modulated pulse pattern, in continuous or cyclical burst mode) is experimented per individual, clinically determined and then pre-programmed into the patient's intraoral Stimulator device by the patient's sleep medicine physician at the time of evaluation and testing of the Stimulator device assembly in the patient. The physician uses the external (inductive) Programmer appliance in close proximity to the intraoral Stimulator device assembly in-situ (i.e. in the patient's mouth) for electro-magnetic coupling. The parameters in the implanted intraoral device cannot be changed by the patient, but the device can be turned on or off by the patient at the sleep and wake times, by bringing the Recharger appliance into close proximity to the Stimulator device assembly. In the basic embodiment of the invention without the sensors, the stimulation therapy as programmed and setup by the physician starts as soon as the patient turns On the device at sleep time and the therapy stops as soon as the patient turns Off the device when awake, without regard to the respiratory cycle or the muscle tone of the genioglossus muscle. This is a normal open loop operation.

In other embodiments, a closed loop operation is implemented whereby the stimulation therapy is in some way changed, modulated or synchronized to respiratory activity or muscle activity or patient activity, by using the information conveyed by one or more sensors such as accelerometer, temperature, piezoelectric film and EMG. In the 3-axis MEMS (Micro Electro-Mechanical Systems) Accelerometer sensor sub-system 909 in FIG. 9, the accelerometer detects the patient position and activity. This information may then be used by the main micro-controller for qualifying the onset/offset of therapy or changing the therapy modality; if the accelerometer indicates that the patient is lying on his side, possibly the tongue drooping to that side, perhaps the axis of stimulation current vector can be changed accordingly by selecting the appropriate electrode pair. The accelerometer is housed inside the Stimulator housing.

In the embodiments implementing a thermistor type or semiconductor diode type Temperature Sensor sub-system 910 in FIG. 9, the temperature reading can indicate if the intraoral device assembly is actually inside the mouth or outside the mouth; if it is outside the mouth for more than certain time, perhaps the micro-controller can judiciously turn Off the device to conserve battery power. The temperature sensor may be housed inside the Stimulator device housing or outside on the oral housing, with proper water-tightness in that case.

In the embodiments implementing a Piezoelectric Film Sensor sub-system 911 in FIG. 9, the sensor can be used for possibly detecting the respiratory movements in the oral cavity. Piezoelectric film produces a voltage in proportion to compressive or tensile mechanical stress or strain, making it an ideal dynamic strain gage. The sensor can be housed at an appropriate location on the oral housing of the Stimulator assembly or part of the electrode assembly, with proper water-tightness. The sensor information may be analyzed by the micro-controller and may be used for synchronizing the stimulation therapy to the patient's intrinsic respiratory activity.

In the embodiments implementing an EMG (electromyogram) Acquisition & Detection sub-system 912 in FIG. 9, using one of the electrode pairs as the EMG sensing electrodes, the EMG activity of the genioglossus muscle may be used to determine if the muscle has lost the tone during sleep. Every time the Stimulator device is turned on for stimulation therapy before sleep, the EMG sub-system can briefly acquire the electromyogram signal of the genioglossus muscle between the electrodes and use that as baseline or wake-state signal. During sleep therapy, the periodically acquired EMG can be compared against the baseline signal level; if the sleep time EMG is found to be reduced or lost compared to the wake-state or baseline activity level, then that detection information can be used by the micro-controller for initiating or synchronizing the stimulation.

The external (inductive) Recharger Appliance is a mains-operated small portable device with a rechargeable battery backup that has the inductive energy transmission circuit and a large built-in electrical coil for inductively coupling the power energy to the receiver coil in the intraoral Stimulator device assembly. The Recharger is generally expected to be left plugged into the ac mains and kept on the patient's bedside table. In order to recharge the rechargeable battery in the intraoral Stimulator device assembly, the device assembly needs to be placed in the cradle provided of the Recharger appliance and charging needs to be initiated by the patient. A completely depleted battery in the intraoral Stimulator device may take a few hours to charge to full capacity. While charging and thereafter, the battery status of the intraoral device battery is indicated on the Recharger appliance user-interface, by means of the Recharger receiving that data information periodically through the data telemetry from the intraoral device.

The same Recharger is also used by the patient for turning On or Off the intraoral Stimulator device at sleep and wake times, which the patient must do so by bringing the Stimulator device assembly and the Recharger in close proximity of each other.

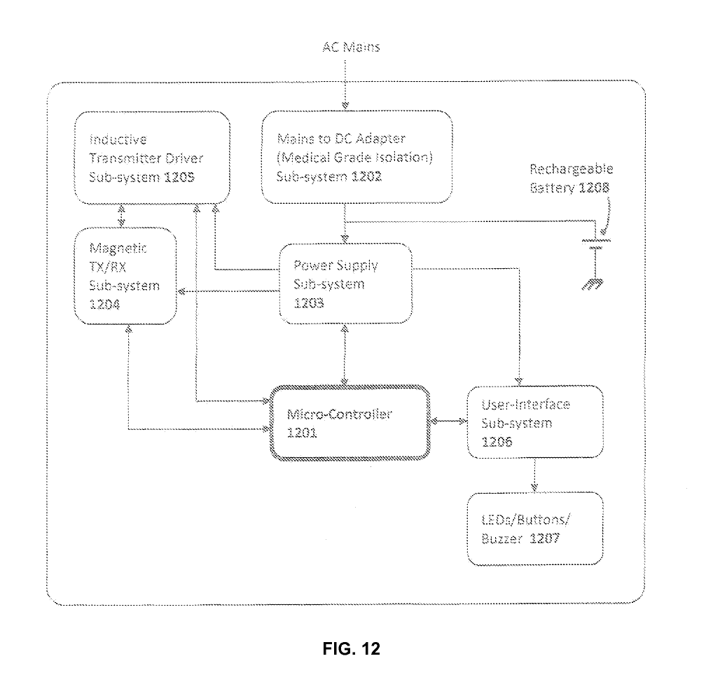

As shown in FIG. 12, functional sub-systems of the Recharger appliance are as follows, and are easily recognizable by someone familiar in the art of electromagnetic induction for energy transmission and half-duplex data communication in implantable medical devices: Micro-Controller 1201, Mains to DC Adapter (Medical Grade Isolation) sub-system 1202, Power-Supply sub-system 1203, Magnetic Transmit/Receive (TX/RX) sub-system 1204, Inductive Transmitter Driver sub-system 1205, User-Interface sub-system 1206, LEDs/Buttons/Buzzer 1207 and a Rechargeable Battery 1208. The intelligence of the Recharger system lies in the firmware executing in the micro-controller, as the system is a software (firmware) controlled hardware system. The Mains to DC Adapter (with medical grade Isolation) sub-system 1202 provides the required DC voltage to the system while charging the Rechargeable Battery 1208; the Recharger can be operated from its charged battery without the ac mains connection, if necessary. Power-Supply sub-system 1203 provides the required power supply voltages for the entire system, including the high voltage required for the Inductive Transmitter Driver sub-system 1205. The Magnetic Tx/Rx sub-system 1204 is the controller for the Inductive Transmitter Driver sub-system 1205 as well as includes the half-duplex data communication circuits. The Inductive Transmitter Driver sub-system 1205 drives the large transmitting inductive coil, which also serves as the receive coil for protocol acknowledgement and battery data coming back from the intraoral Stimulator device assembly. In another embodiment, there may be a separate receive coil. The bi-directional data communication protocol including checksum and error detection is handled by the micro-controller 1201. User-Interface sub-system 1206 is responsible for handling the user inputs through buttons and audio-visual feedback to the user through LEDs and buzzer (LEDs/Buttons/Buzzer 1207). Buttons are provided on the face of the Recharger appliance for the user to initiate charging of the intraoral Stimulator device, and to turn On/Off the intraoral device during sleep and wake times.

The external (inductive) Programmer Appliance is a battery-operated hand-held small portable device that has the inductive data transmission and receiver circuit and a large built-in electrical coil for inductively coupling the data transmission energy to the receiver coil in the intraoral Stimulator device assembly. A Programmer is given to each patient's sleep medicine physician and it enables the physician to remotely adjust or program the stimulation parameters and other device parameters in the intraoral Stimulator devices of the physician's patients, at the time of initial evaluation and device installation, and at subsequent follow up visits. A simple menu driven system with hard and/or soft buttons is implemented in the Programmer appliance to enable the physician to select the parameters and transmit the parameters to the intraoral Stimulator device through an (inductive) bidirectional half-duplex transmission schema similar to the one used in the Recharger appliance, but without power transmission for battery charging. In order to change the parameters in the intraoral device, the Programmer needs to be in close proximity to the intraoral Stimulator device in-situ, which is generally expected to be in the mouth. The Programmer operates with a primary battery and is not ac mains operated.

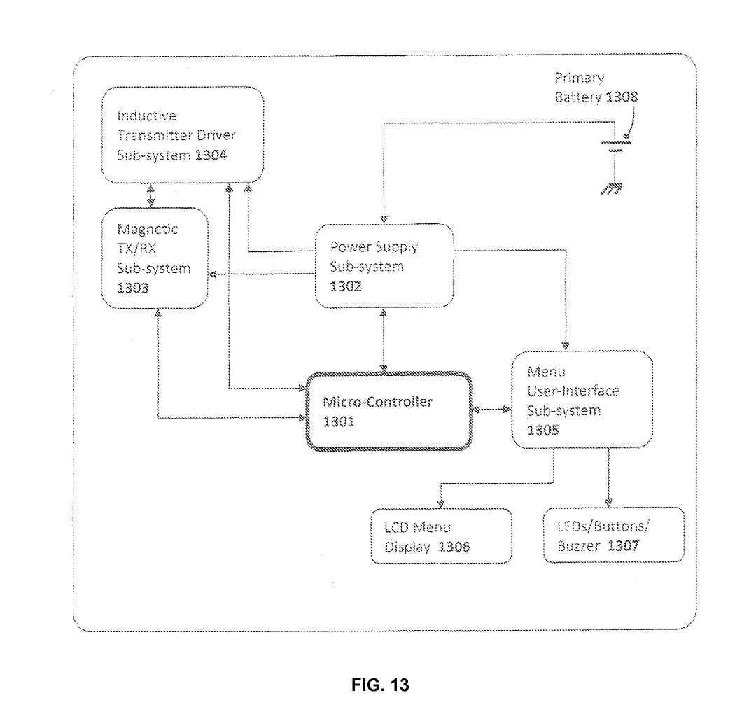

As shown in FIG. 13, functional sub-systems of the Programmer appliance are as follows, and are easily recognizable by someone familiar with the art of general Pacemaker Programmers with inductive half-duplex data communication: Micro-Controller 1301, Power-Supply sub-system 1302, Magnetic Transmit/receive Tx/Rx sub-system 1303, Inductive Transmitter Driver sub-system 1304, Menu User-Interface sub-system 1305, LCD Menu Display 1306, LEDs/Buttons/Buzzer 1307, and a Primary Battery (non-rechargeable) 1308. The intelligence of the Recharger system lies in the firmware executing in the micro-controller, as the system is a software (firmware) controlled hardware system. The Programmer is operated from the Primary Battery 1308, and does not connect to the ac mains. Power-Supply sub-system 1302 provides the required power supply voltages for the entire system, including the high voltage required for the Inductive Transmitter Driver sub-system 1304. The Magnetic Tx/Rx sub-system 1303 is a half-duplex data communication system as well as the controller for the Inductive Transmitter Driver sub-system 1304. The Inductive Transmitter Driver sub-system 1304 drives the large transmitting inductive coil, which also serves as the receive coil for protocol acknowledgement and data coming back from the intraoral Stimulator device assembly. In another embodiment, there may be a separate receive coil. The bi-directional data communication protocol including checksum and error detection is handled by the micro-controller 1301. Menu User-Interface sub-system 1305 is responsible for handling the user inputs through buttons and the audio-visual feedback to the user through LEDs and buzzer (LEDs/Buttons/Buzzer 1307), and displaying and controlling the parameter menus on LCD Menu Display 1306. Buttons are provided on the Programmer appliance for the user to initiate communication with the intraoral Stimulator device.

What is claimed is:

1. A non-invasive intraoral electrical Stimulator or Pacemaker system and method for the therapeutic, prophylactic or preventative treatment of Obstructive Sleep Apnea (OSA) in human adults and young adults, consisting of a removable intraoral Stimulator or Pacemaker device assembly, an external inductive Recharger appliance and an external hand-held inductive Programmer appliance, with the following claims of features:
the intraoral Stimulator device assembly is worn on the lower teeth in the mouth by the OSA patient before sleep time and is removed when awake or during normal activity, and cleaned with medical wipes; and
the intraoral Stimulator device assembly consists of a rechargeable battery-powered electrical Stimulator device (a.k.a. pulse generator) with one or more micro-controllers, inductive coupling coils, one or more sensors and electronic sub-systems for the primary purpose of generation and control of stimulation current or voltage pulses for the excitation and contraction of tongue protruder muscles, primarily the genioglossus and other associated tongue muscles, preferentially in the posterior sublingual or base-of-tongue region; and
the intraoral Stimulator device as above, is assembled and enclosed in a hermetically sealed encapsulation or housing of a biocompatible material such as titanium, stainless steel, epoxy, silicone, polyurethane, glass ionomer, thermoplastic, or thermosetting polymers; and the encapsulation or housing is over-coated with a soft non-abrasive dental or biocompatible polymer material to be comfortable in the mouth; and
the intraoral Stimulator device assembly consists of up to two pairs of electrodes that are positioned ventral-laterally at the sublingual posterior to middle genioglossus muscle or base-of-tongue for either bilateral stimulation (i.e. across sides) or unilateral stimulation (i.e. on one side) of the above mentioned muscles and also for acquiring the electromyographic (EMG) signal of the genioglossus muscle; and
the intraoral Stimulator device in the encapsulation or housing and the bilateral or unilateral electrodes as above are connected together by leads and are integrally embedded into a removable hollow dental wire-frame housing or dental oral molded housing covering full or partial length of the lower teeth or embedded into a molar teeth clip; and
the intraoral Stimulator device contains a tuned electrical coil and a charge converter, for inductively coupling to the transmitted power from an external Recharger appliance, and converting that coupled energy to a voltage for charging the rechargeable battery in the device; and the same or another tuned electrical coil for bidirectional or half-duplex data communication between the intraoral Stimulator and the external transmitter sources; and
an external (to the human body) inductive Recharger appliance with an electric coil for charging the rechargeable battery in the intraoral Stimulator device, by transferring charging energy or power and data through an electro-magnetic induction or transmission schema; and
an external (to the human body) inductive Programmer appliance utilizing an electric coil and data transmission schema by electromagnetic induction, for enabling the patient's physician to program the stimulation and system parameter data in the intraoral Stimulator device.

2. The system and method of claim 1 wherein the Stimulator device contains a rechargeable battery of lithium-ion, or thin film lithium-ion or lithium polymer chemistry; and a rechargeable power receiver sub-system for charging the rechargeable battery from the inductively-coupled received energy.

3. The system and method of claim 1 wherein the Stimulator device contains an electronic Stimulation Pulse Generator sub-system that generates up to two independent channels of symmetrical and/or asymmetrical biphasic stimulation pulses for charge balance, of amplitude 0 to 15 mA, pulse widths of 100 to 1000 uSec, pulse repetition rates of 5 to 150 pulses per second (pps) and inter-channel delay of 0 to 500 mSec, deliverable into a muscle and nerve load of 200-2500 ohms.

4. The system and method of claim 3 wherein the Stimulation Pulse Generator is programmed to generate the stimulation pulses normally at a fixed pulse amplitude, pulse width and pulse repetition rate or programmed to modulate or vary any one or more of these parameters automatically in a pre-defined cyclical pattern, including initial gradual step-up in pulse amplitude:
whether normal or modulation pattern, the generator is further programmed to generate the pattern continuously for a programmed time duration of 1 minute to 12 hours; and
the generator is programmed to generate cyclical burst or turn ON the pattern for a time duration of 1 second to 2 minutes and turn OFF the pattern for a time duration of 1 second to 2 minutes, and repeat the cycle continuously or for a total time duration of 4 minutes to 12 hours.

5. The system and method of claim 1 wherein, in a basic or standard embodiment, the Stimulator device works in an open loop configuration, namely when the patient turns ON the stimulation therapy before falling asleep, the stimulation is started after a fixed delay without regard to the patient's respiration cycle or activity; and the stimulation is turned off immediately or after a delay when the patient turns OFF the therapy.

6. In advanced embodiments, the system and method of claim 1 wherein the Stimulator device works in a closed loop configuration, namely the stimulation is regulated based on the information provided by one or more sensors in the device as under:
a 3-axis MEMS accelerometer sensor sub-system for detecting the patient activity and/or position, for determining the wakeful or sleep state of the patient and using that data to qualify, modulate or turn ON/OFF the stimulation therapy; and/or
a temperature sensor sub-system, such as a thermistor or a semiconductor diode for determining if the intraoral device is inside the mouth or outside the mouth, and use that information for power control ON/OFF of the Stimulator device; and/or
an electromyogram (EMG) sub-system for the acquisition and detection of electro-myographic activity of the genioglossus muscle between any one pair of electrodes, and use that information for the regulation of the stimulation therapy in the Stimulator device; and/or
a piezoelectric film sensor sub-system for the detection of respiratory cycle activity and/or tongue muscle movement in the mouth, and use that information for the regulation of the stimulation therapy in the Stimulator device.

7. The system and method of claim 1 wherein the Stimulator device contains an electrode interface sub-system for switching and time-multiplexing the single or dual channel stimulation outputs to the one or two bilateral electrode pairs or a single unilateral electrode pair, for establishing bilateral or unilateral electrical vector of stimulation in the sublingual genioglossus or under-the-tongue muscle strata.

8. The system and method of claim 1 wherein the electrodes are made of Silver-SilverChloride (Ag—AgCl) or Platinum-Iridium (Pt—Ir 90/10) or gold plated silver material, and are of solid or hollow spherical shape of 2-10 mm diameter or cigar shape of 2-10 mm diameter & 5-10 mm length or pancake shape of 8-40mm circumference and 2-10 mm thickness, and of textured surface for increased surface area.

9. The system and method of claim 1 wherein the Stimulator device and the electrodes are connected together by platinum, MP35N or silver conductor wire, straight or coiled, insulated with silicone, polyurethane or a fluoropolymer jacket and integrally embedded in the dental wire-frame housing, dental oral molded housing or dental molar teeth clip.

10. The system and method of claim 1 wherein the removable dental oral housing appliance or the molar teeth clip over the lower teeth, is a custom-made (per patient) assembly fabricated utilizing a replicate model of lower teeth structure by the conventional dental impression methods, and a thermal over-mold forming process utilizing conventional dental materials, polymeric and stainless steel wire frame for reinforcement.

11. The system and method of claim 10 wherein the dental wire-frame or molded oral housing of the Stimulator device for bilateral stimulation engages almost all lower teeth or some lower teeth consisting of at least one molar tooth and/or the gingival tissue surrounding the molars and one or more other teeth particularly the incisor teeth on both sides of the mouth, and is constructed for easy installation into and removal from the mouth much like a dental mouth-guard or retainer.

12. The system and method of claim 10 wherein the molar teeth clip of the Stimulator device for unilateral stimulation engages one or more lower molar teeth and/or the gingival tissue surrounding the molars, and is constructed for easy installation into and removal from the mouth.

13. The system and method of claim 1 wherein the encapsulation or housing of the Stimulator device is smoothly shaped, profiled and integrated with the dental wire-frame, molded dental oral housing or molar teeth clip, so it is positioned preferably between the teeth and either cheek or in the space between the mouth pallet and the tongue, without interfering with the bite; and the encapsulation or housing is over-coated with soft dental material or silicone for gentle feel in the mouth.

14. The system and method of claim 1 wherein the Stimulator device contains an inductive data communication telemetry sub-system for receiving and transmitting stimulation control parameters and system data through the same electromagnetic induction transmission as used for device battery charging.

15. The system and method of claim 1 wherein the external inductive Recharger appliance consists of a micro-controller, an electronic tuned charging circuit and an electromagnetic inductive transmission schema, for coupling the energy to the magnetic receiver circuit in the intraoral Stimulator device when in near proximity and also for bidirectional half-duplex data communication, as under:
the Recharger appliance consists of a large electrical coil for inductive transmit/receive coupling with the intraoral Stimulator assembly; and
the intraoral Stimulator assembly is removed from the mouth and placed in the cradle of the Recharger appliance (so the Stimulator assembly is in close proximity), for charging the rechargeable battery in the intraoral Stimulator device; and
the Recharger appliance operates from ac mains through a medical grade isolation dc adapter or battery charger, and contains an integrated backup rechargeable Li-ion battery so the appliance can still be fully functional even when unplugged from the ac mains; and
the Recharger appliance is also used for turning on or off the therapy in the intraoral Stimulator device, and for indicating the status of the rechargeable battery of the intraoral Stimulator device.

16. The system and method of claim 1 wherein the external hand-held inductive Programmer appliance consists of a micro-controller, an electronic tuned charging circuit and an electromagnetic inductive transmission schema, for bidirectional half-duplex data communication with the intraoral Stimulator assembly when in close proximity, as under:.
the Programmer appliance consists of a large electrical coil pad with extendable cable for inductive transmit/receive coupling with the intraoral Stimulator assembly; and
the Programmer appliance or just the coil pad is brought in close proximity to the intraoral Stimulator assembly in the patient's mouth, for communicating and programming the stimulation parameters and other system parameters in the intraoral Stimulator assembly; and
the Programmer appliance operates from a primary battery or a rechargeable battery that is removed and recharged separately, so there is no ac mains connection to the Programmer; and
the Programmer appliance is a micro-controller firmware driven menu-based user-interface system consisting of hard buttons, soft buttons and display that enables the patient's physician to select the stimulation mode and parameters and allows programming of these parameters in the intraoral Stimulator device.
